(19) United States
(12) Patent Application Publication
(10) Pub. No.: US 2014/0174425 A1
(43) Pub. Date: Jun. 26, 2014

(54) Easy Warmers

(71) Applicant: Bruce Burks, Gardendale, AL (US)

(72) Inventor: Bruce Burks, Gardendale, AL (US)

(21) Appl. No.: 13/721,341

(22) Filed: Dec. 20, 2012

(51) Int. Cl. A61F 7/08

(52) U.S. Cl. CPC A61F 7/08
USPC 126/204

(57) ABSTRACT

A toe warmer assembly attaches to a foot to warm toes directly or through footwear. The assembly includes a pouch having a closed end, an open end, and a perimeter wall extending from the closed end to the open end defining an interior space. A flap extends from the open end of the pouch and is selectively positioned extending over the open end wherein the open end is obstructed by the flap. A first strap has a first end coupled to the pouch and a second end coupled to the pouch wherein the first strap forms a first loop extending from the pouch. An exothermic member is positioned in the interior space of the pouch wherein the exothermic member delivers heat through the pouch.

### BACKGROUND OF THE DISCLOSURE

### Field of the Disclosure

The disclosure relates to warming devices and more particularly pertains to a new warming device for attaching to a foot to warm toes directly or through footwear.

### SUMMARY OF THE DISCLOSURE

An embodiment of the disclosure meets the needs presented above by generally comprising a pouch having a closed end, an open end, and a perimeter wall extending from the closed end to the open end defining an interior space. A flap extends from the open end of the pouch and is selectively positioned extending over the open end wherein the open end is obstructed by the flap. A first strap has a first end coupled to the pouch and a second end coupled to the pouch wherein the first strap forms a first loop extending from the pouch. An exothermic member is positioned in the interior space of the pouch wherein the exothermic member delivers heat through the pouch.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a partially exploded top front side perspective view of a toe warmer assembly according to an embodiment of the disclosure.

FIG. 2 is a front view of an embodiment of the disclosure.

FIG. 3 is a bottom view of an embodiment of the disclosure.

FIG. 4 is a cross-sectional view of an embodiment of the disclosure taken along line 4-4 of FIG. 1.

FIG. 5 is a side view of an embodiment of the disclosure in use.

### DESCRIPTION OF THE PREFERRED EMBODIMENT

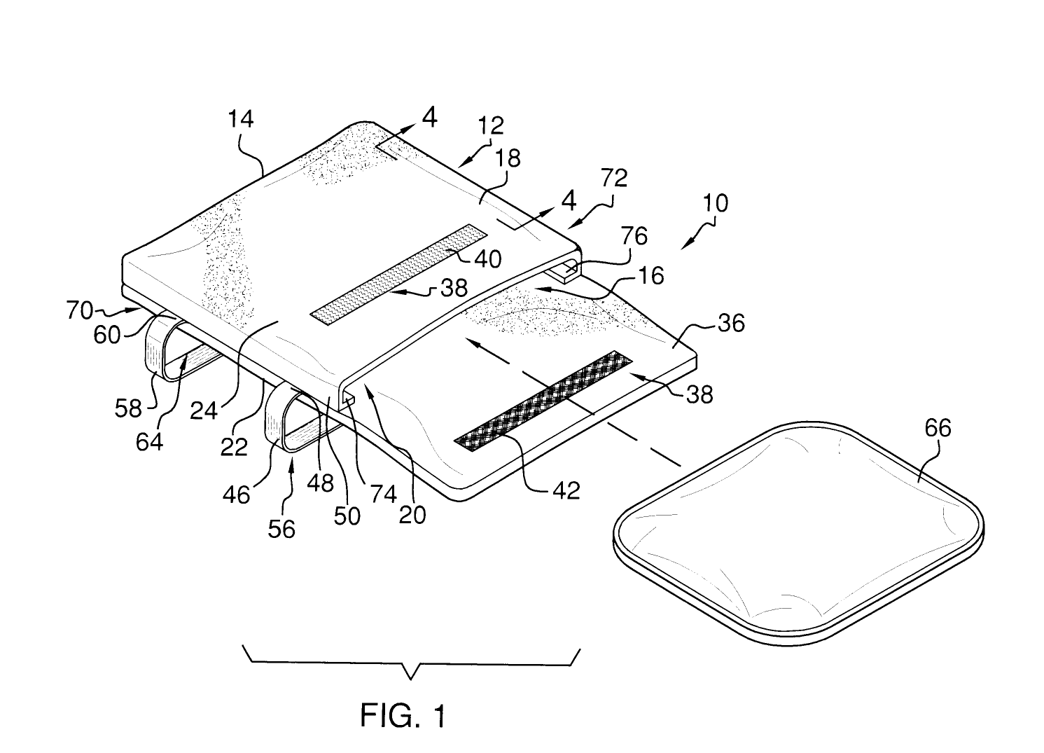

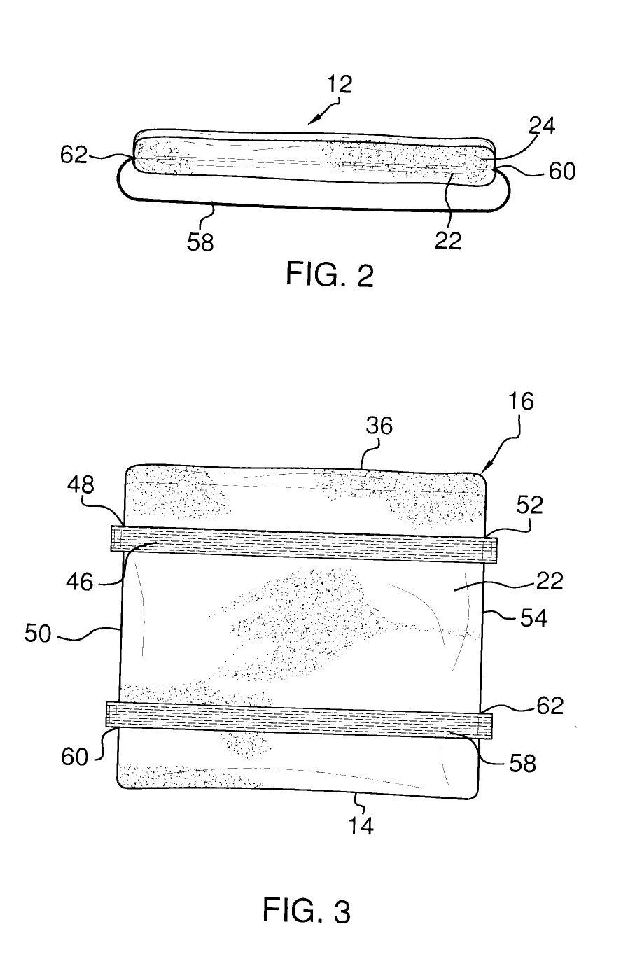

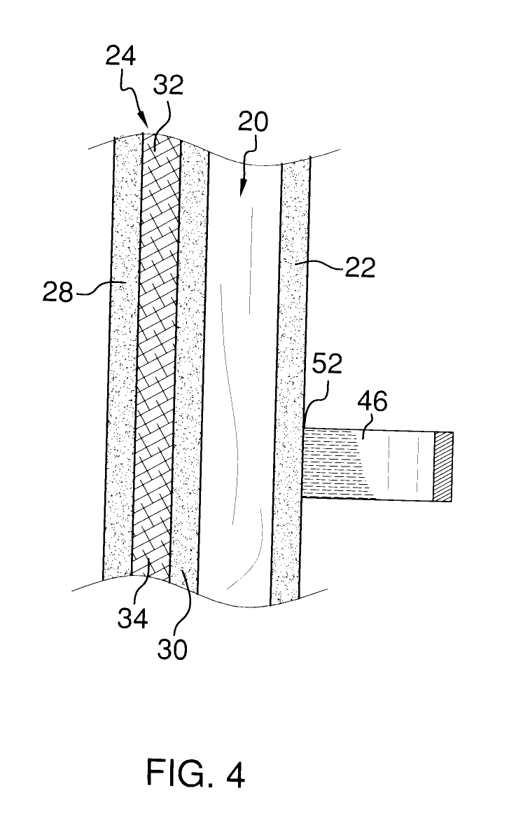

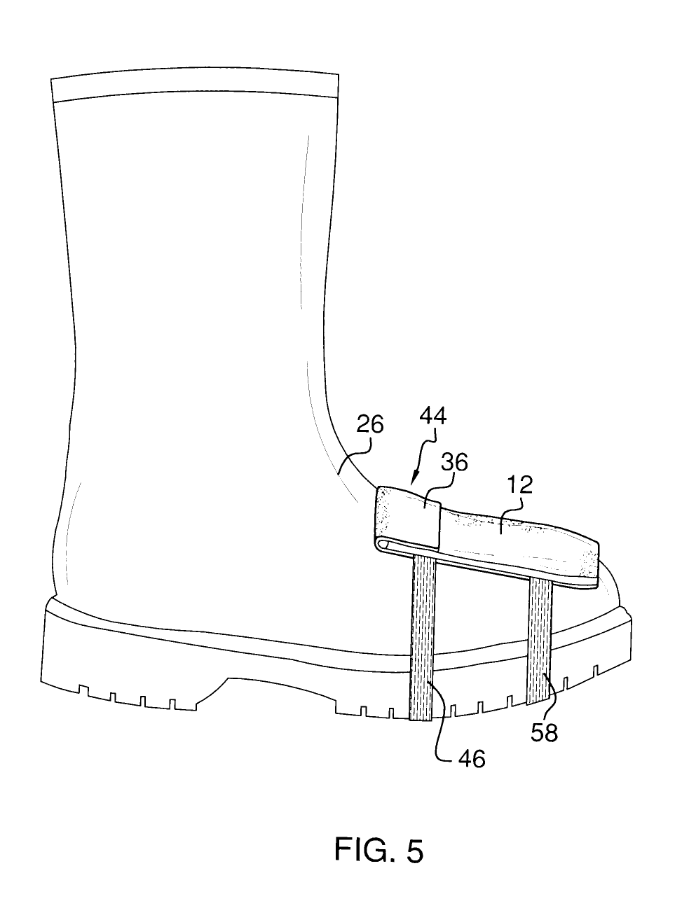

With reference now to the drawings, and in particular to FIGS. 1 through 5 thereof, a new warming device embodying the principles and concepts of an embodiment of the disclosure and generally designated by the reference numeral 10 will be described.

As best illustrated in FIGS. 1 through 5, the toe warmer assembly 10 generally comprises a pouch 12 having a closed end 14, an open end 16, and a perimeter wall 18 extending from the closed end 14 to the open end 16 defining an interior space 20. The perimeter wall 18 has an inward side 22 positioned to abut a foot or footwear 26 and an outward side 24 facing away from the footwear 26. The inward side 22 may have a single layer of material such as fleece to promote transfer of heat from the interior space 20. The outward side 24 may have a pair of outer layers 28,30 of the fleece material and a medial layer 32 positioned between the outer layers 28,30. The medial layer 32 is constructed of an insulating material 34 to inhibit heat transfer from the interior space 20 outwardly away from the footwear 26. The outward side 24 may extend around lateral sides 70,72 of the pouch 12 having edge portions 74,76 attached to the inward side 22.

A flap 36 extends from the open end 16 of the pouch 12. The flap 36 may be an integral extension of the perimeter wall 18. The flap 36 is selectively positioned extending over the open end 16 wherein the open end 16 is obstructed by the flap 36. A closure 38 has a first section 40 coupled to the pouch 12 and a second section 42 coupled to the flap 36 wherein the flap 36 is selectively coupled to the pouch 12 in a closed position 44 extending over the open end 16 of the pouch 12. The closure 38 may comprise hook and loop fastener, snaps, or the like.

A first strap 46 has a first end 48 coupled to the perimeter wall 18 of the pouch 12 along a first lateral edge 50 and a second end 52 coupled to the perimeter wall 18 along a second lateral edge 54 of the pouch 12 wherein the first strap 46 forms a first loop 56 extending from the pouch 12. The first strap 46 is positioned proximate the open end 16 of the pouch 12. The first strap 46 is resiliently elastic along a length of the first strap 46 to facilitate attachment of the pouch 12 to the footwear 26. A second strap 58 has a first end 60 coupled to the first lateral edge 50 of the perimeter wall 18 of the pouch 12 and a second end 62 coupled to the second lateral edge 54 of the perimeter wall 18 of the pouch 12 wherein the second strap 58 forms a second loop 64 extending from the pouch 12. The second strap 58 is positioned proximate the closed end 14 of the pouch 12. The second strap 58 is also resiliently elastic along a length of the second strap 58 to facilitate attachment of the pouch 12 to the foot or footwear 26. The first strap 46 and the second strap 58 are positioned in spaced parallel relationship to each other with each extending transversely from the pouch 12 relative to the open end 16 of the pouch 12.

An exothermic member 66 such as the type activate by agitation of the contents of a packet is positioned in the interior space 20 of the pouch 12. The exothermic member 66 may also comprise a self-contained battery and heating element. The exothermic member 66 is held within the interior space 20 where it delivers heat through the pouch 12, particularly the inward side 22 of the pouch 12.

In use, the pouch 12 is attached to the foot or footwear 26 using the first strap 46 and second strap 58. The exothermic member 66 is activated and positioned in the interior space 20. The flap 36 is secured in the closed position 44 to hold the exothermic member 66 within the pouch 12 where it delivers heat directed towards the foot or footwear 26.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure.

I claim:

1. A toe warming assembly comprising:
a pouch having a closed end, an open end, and a perimeter wall extending from said closed end to said open end defining an interior space;
a flap extending from said open end of said pouch, said flap being selectively positioned extending over said open end wherein said open end is obstructed by said flap;
a first strap having a first end coupled to said pouch and a second end coupled to said pouch wherein said first strap forms a first loop extending from said pouch;
an exothermic member positioned in said interior space of said pouch wherein said exothermic member delivers heat through said pouch.

2. The assembly of claim 1, further comprising said first strap being resiliently elastic along a length of said strap.

3. The assembly of claim 1, further comprising a second strap having a first end coupled to said pouch and a second end coupled to said pouch wherein said second strap forms a second loop extending from said pouch.

4. The assembly of claim 3, further comprising said second strap being resiliently elastic along a length of said second strap.

5. The assembly of claim 3, further comprising said second strap being positioned proximate said closed end of said pouch.

6. The assembly of claim 5, further comprising said first strap being positioned proximate said open end of said pouch.

7. The assembly of claim 6, further comprising said first strap and said second strap each extending transversely from said pouch relative to said open end of said pouch.

8. The assembly of claim 1, further comprising a closure coupled to said pouch and said flap wherein said flap is selectively coupled to said pouch in a closed position extending over said open end of said pouch.

9. The assembly of claim 1, further comprising said perimeter wall having an inward side and an outward side, said outward side having a pair of outer layers and a medial layer positioned between said outer layers, said medial layer being an insulating material.

10. A toe warming assembly comprising:
a pouch having a closed end, an open end, and a perimeter wall extending from said closed end to said open end defining an interior space, said perimeter wall having an inward side and an outward side, said outward side having a pair of outer layers and a medial layer positioned between said outer layers, said medial layer being an insulating material;
a flap extending from said open end of said pouch, said flap being selectively positioned extending over said open end wherein said open end is obstructed by said flap;
a first strap having a first end coupled to said pouch and a second end coupled to said pouch wherein said first strap forms a first loop extending from said pouch, said first strap being positioned proximate said open end of said pouch, said first strap being resiliently elastic along a length of said strap;
a second strap having a first end coupled to said pouch and a second end coupled to said pouch wherein said second strap forms a second loop extending from said pouch, said second strap being positioned proximate said closed end of said pouch, said second strap being resiliently elastic along a length of said second strap, said first strap and said second strap each extending transversely from said pouch relative to said open end of said pouch;
an exothermic member positioned in said interior space of said pouch wherein said exothermic member delivers heat through said pouch; and
a closure having a first section coupled to said pouch and a second section coupled to said flap wherein said flap is selectively coupled to said pouch in a closed position extending over said open end of said pouch.
